(19) United States
(12) Patent Application Publication
(10) Pub. No.: US 2026/0282170 A1
(43) Pub. Date: Sep. 17, 2026

(54) TWO PART HEATER ASSEMBLY

(71) Applicant: Philip Morris Products S.A., Neuchatel (CH)

(72) Inventors: Jérôme Christian COURBAT, Cortaillod (CH); Leander DITTMANN, Crissier (CH); Ross Peter JONES, Cambridge (GB)

(73) Assignee: Philip Morris Products S.A., Neuchatel (CH)

(21) Appl. No.: 19/167,979

(22) PCT Filed: Mar. 28, 2024
(86) PCT No.: PCT/EP2024/058638

§ 371 Date: Sep. 23, 2025

(30) Foreign Application Priority Data
Mar. 29, 2023 (EP) 23165222.3

(51) Int. Cl. H05B 3/14; A24F 40/10; A24F 40/44; A24F 40/46; A24F 40/70; H05B 3/06; H05B 3/26

(52) U.S. Cl. CPC H05B 3/141; A24F 40/10; A24F 40/44; A24F 40/46; A24F 40/70; H05B 3/06; H05B 3/265; H05B 2203/013; H05B 2203/017; H05B 2203/021

(57) ABSTRACT

A heater assembly for an aerosol-generating system, the heater assembly including: a heating element configured to vaporise a liquid aerosol-forming substrate; and a porous ceramic body configured to convey the liquid aerosol-forming substrate to the heating element, the porous ceramic body having a liquid absorption surface and a heating surface, the heating element being located on and bonded to the heating surface of the porous ceramic body.

The present disclosure relates to a heater assembly for an aerosol-generating system. In particular, but not exclusively, the present disclosure relates to a heater assembly for a handheld electrically operated aerosol-generating system for heating an aerosol-forming substrate to generate an aerosol and for delivering the aerosol into the mouth of a user. The present disclosure further relates to a cartridge and an aerosol-generating system comprising the heater assembly and also to method of manufacturing a heater assembly.

Aerosol-generating systems that heat a liquid aerosol-forming substrate in order to generate an aerosol for delivery to a user are generally known in the prior art. These systems typically comprise an aerosol-generating device and a replaceable cartridge. The cartridge includes a liquid aerosol-forming substrate that is capable of releasing volatile compounds when heated. The cartridge typically also includes a heater for heating the liquid aerosol-forming substrate. In known aerosol-generating systems, the heater comprises a resistive heating element wound around a wick that supplies liquid aerosol-forming substrate to the heating element. The aerosol-generating device or cartridge also comprises a mouthpiece. When a user takes a puff on the mouthpiece, an electric current is passed through the heating element causing it to be heated by resistive or Joule heating, which, in turn, heats the liquid aerosol-forming substrate supplied by the wick. This causes volatile compounds to be released from the liquid aerosol-forming substrate that cool to form an aerosol. The aerosol is then drawn into a user's mouth via the mouthpiece.

In some other known aerosol-generating systems, the aerosol-generating system comprises a heater assembly having a resistive heating element located on a heating surface of a porous body. Liquid aerosol-forming substrate is supplied from a liquid storage portion to the heating element via the pores of the porous body by capillary action. Such known aerosol-generating systems have a number of drawbacks. For example, they can be difficult to manufacture with consistent manufacturing tolerances which can result in inconsistent vapour production and flavour generation. Inconsistent manufacturing tolerances can also affect the transfer of heat from the heating element to the wick reducing the energy efficiencies of such devices. A further problem encountered by such known aerosol-generating systems is “dry heating” or a “dry puff”, which arises when the heating element is heated with insufficient liquid aerosol-forming substrate being supplied to the heating element. This can occur, for example, when a user has consumed all of the liquid aerosol-forming substrate in the cartridge such that the cartridge is depleted of liquid aerosol-forming substrate and needs replacing. During operation, it is preferable to maintain a supply of liquid aerosol-forming substrate to the heating element such that the heating element is maintained in a wet state because this helps to ensure that a satisfactory aerosol is produced when a negative pressure is applied at the mouthpiece. Dry heating can result in overheating of the heating element and, potentially, thermal decomposition of the liquid aerosol-forming substrate, which can produce undesirable by-products and an unsatisfactory aerosol. Allowing the aerosol-generating system to continue to operate when liquid aerosol-forming substrate is not being supplied to the heating element can result in a poor user experience.

A number of prior art documents disclose aerosol-generating systems having a porous transport element and a separate heating element, both of which are assembled within the aerosol-generating system such that the heating element, to which power is supplied through electrical contacts, heats the porous transport element. Such known systems can be difficult to manufacture and assemble with consistent manufacturing tolerances which can result in inconsistent vapour production and flavour generation. Inconsistent manufacturing tolerances can also affect the transfer of heat from the heating element to the porous transport element reducing the energy efficiencies of such systems. Liquid is supplied from a liquid reservoir to the heating element via pores within the transport element. This known aerosol-generating system may also experience a “dry heating” or “dry puff” situation, and as such has the associated disadvantages, namely undesirable by-products, an unsatisfactory aerosol, and a poor user experience.

It would be desirable to provide a heater assembly which is easier to reliably manufacture and assemble, thus resulting in a more energy efficient heater assembly capable of generating a more consistent aerosol. It would be desirable to provide a heater assembly that reduces the likelihood of a user experiencing dry heating or a dry puff.

The present disclosure relates to a heater assembly for an aerosol-generating system. The heater assembly may comprise a heating element for vaporising a liquid aerosol-forming substrate. The heater assembly may comprise a porous ceramic body for conveying the liquid aerosol-forming substrate to the heating element. The porous ceramic body may have a liquid absorption surface and a heating surface. The heating element may be located on and bonded to the heating surface of the porous ceramic body.

According to the present invention, there is provided a heater assembly for an aerosol-generating system. The heater assembly comprises a heating element for vaporising a liquid aerosol-forming substrate. The heater assembly comprises a porous ceramic body for conveying the liquid aerosol-forming substrate to the heating element. The porous ceramic body has a liquid absorption surface and a heating surface. The heating element is located on and bonded to the heating surface of the porous ceramic body.

As used herein, the term “aerosol-generating device” relates to a device that interacts with a liquid aerosol-forming substrate to generate an aerosol.

As used herein, the term “aerosol-generating cartridge” relates to a component that interacts with a liquid aerosol-forming device to generate an aerosol. An aerosol-generating cartridge contains, or is configured to contain, a liquid aerosol-generating substrate.

As used herein, the term “liquid aerosol-generating substrate” relates to a liquid substrate capable of releasing volatile compounds that can form an aerosol. Such volatile compounds can be released by heating the aerosol-forming substrate.

As used herein, the term “heating element” refers to a component which transfers heat energy to the liquid aerosol-generating substrate. It will be appreciated that the electrical heating element may be deposited directly on the porous body.

As used herein, the term “porous body” refers to a component which has a plurality of pores, at least some of which are interconnected. The porous body is configured to contain liquid within the plurality of pores.

As used herein, the term “thermally insulating” refers to a property in which heat transfer is reduced or restricted. A more thermally insulating component will transfer less heat, via conduction, convection or radiation, than a more thermally insulating component.

The heater assembly of the present invention provides an improved component for an aerosol-generating system. By providing a heater assembly in which a heating element is on and bonded to a heating surface of a porous ceramic body, a more robust and reliable connection can be established between the heating element and the porous ceramic body. This may advantageously help to improve the transfer of heat between the heating element and the porous ceramic body.

The bonding of the heating element to the heating surface of the porous ceramic body may also advantageously provide a heater assembly which is easier to reliably manufacture and assemble, thus resulting in a more energy efficient heater assembly capable of generating a more consistent aerosol. This, in turn, may provide a user of the aerosol-generating system with an improved and more enjoyable experience. Such an arrangement may also help to reduce the likelihood of a user experiencing dry heating or a dry puff.

An advantage of providing the heating element on and bonded to a heating surface of the porous ceramic body is that it helps to alleviate the problems of manufacturing tolerances encountered with wick and coil heaters and other arrangements in which a heating element is detached from a liquid transport element. The dimensions and arrangement of the electrical heating element relative to the porous body are also fixed, which helps to produce a more consistent aerosol. This is because the electrical heating element is fixed to the porous ceramic body, which helps to supply liquid aerosol-forming substrate to the heating element. This also helps to prevent unwanted loss of heat, which helps to improve energy efficiency.

By providing the heating element on and bonded to a heating surface of the porous ceramic body, the resulting aerosol-generating system may benefit from reduced material requirements. This is because the need for intermediate components which fix the heating element relative to the porous body can be reduced or eliminated entirely. The material savings can result in cost savings of the overall aerosol-generating system. An additional advantage of the reduced material requirements in the overall aerosol-generating system is the provision of a more sustainable and environmentally friendly solution.

The heating element may be fluid permeable.

As used herein, the term “fluid permeable” in the context of the heating element means that liquid aerosol-forming substrate is able to pass from one side of the heating element to the other side of the heating element without needing to go around the heating element.

It will be understood that for the heating element to be fluid permeable, the material from which the heating element is made may be fluid permeable. Alternatively, the material from which the heating element is made may be fluid impermeable, but the structure or arrangement of the heating element may nevertheless allow liquid aerosol-forming substrate to pass from one side of the heating element to the other side of the heating element.

The heating element may be an electrical heating element. For example, the heating element may be a resistive heating element. The heating element may have any suitable shape or form. Examples of suitable shapes and forms include but are not limited to a band, a strip, a filament, a wire, a mesh, a flat spiral coil, fibres or a fabric.

In some preferred examples, the heating element is planar. The planar heating element may extend substantially in a plane.

In some preferred examples, the heating element comprises a mesh. The heating element may comprise an array of filaments forming a mesh. As used herein the term “mesh” encompasses grids and arrays of filaments having spaces therebetween. The term mesh also includes woven and non-woven fabrics.

The filaments may be formed by etching a sheet material, such as a foil. This may be particularly advantageous when the heater assembly comprises an array of parallel filaments.

If the heating element comprises a mesh or fabric of filaments, the filaments may be individually formed and knitted together.

The heating element may comprise an electrically resistive heating element. The heating element may be made from any suitable electrically conductive material. Suitable materials include but are not limited to: semiconductors such as doped ceramics, electrically “conductive” ceramics (such as, for example, molybdenum disilicide), carbon, graphite, metals, metal alloys and composite materials made of a ceramic material and a metallic material. Such composite materials may comprise doped or undoped ceramics. Examples of suitable doped ceramics include doped silicon carbides. Examples of suitable metals include titanium, zirconium, tantalum and metals from the platinum group. Examples of suitable metal alloys include stainless steel, constantan, nickel-, cobalt-, chromium-, aluminum-, titanium-, zirconium-, hafnium-, niobium-, molybdenum-, tantalum-, tungsten-, tin-, gallium-, manganese- and iron-containing alloys, and super-alloys based on nickel, iron, cobalt, stainless steel, Timetal®, iron-aluminum based alloys and iron-manganese-aluminum based alloys. Timetal® is a registered trade mark of Titanium Metals Corporation. The heating element may be made from stainless steel, for example, a 300 series stainless steel such as AISI 304, 316, 304L, 316L. In a preferred example, the electrical heating element May comprise one of more of NiCr and TiZr.

Additionally, the heating element may comprise combinations of the above materials. A combination of materials may be used to improve the control of the resistance of the heating element. For example, materials with a high intrinsic resistance may be combined with materials with a low intrinsic resistance. This may be advantageous if one of the materials is more beneficial from other perspectives, for example price, machinability or other physical and chemical parameters. Advantageously, high resistivity heating allow more efficient use of battery energy.

The porous ceramic body may comprise a porous material having open-cell pores. The plurality of open-cell pores may be interconnected to provide a fluid pathway for aerosol-generating liquid through the porous ceramic body. The porous ceramic body may comprise a material which does not chemically interact with the liquid aerosol-forming substrate. The porous material may have a porosity of between 20 percent and 80 percent. The porous ceramic body may have a flat surface or a curved surface. The porous ceramic body may have a geometrical shape. The porous ceramic body may be in the shape of a cube or a cuboid, or it may have a shape of a disc or a cylinder, or a combination of any of these shapes. The porous ceramic body may comprise or consist of a material with a low thermal conductivity. The porous ceramic body may comprise or consist of non-electrically conductive material. The porous ceramic body may comprise a polymeric or a ceramic material. The porous ceramic body may comprise cotton. The porous ceramic body may comprise porous ceramic, such as but not limited to Al₂O₃, ZrO₂, Si₃N₄, SiC, TiBAlC₂, BN, AlN, SiO₂, MgO, mica, diatomite, silicates, silicides, borides, glass, or a combination of any of these materials. The porous ceramic body may comprise aluminium nitride or silicon carbide. Aluminium nitride and silicon carbide typically have a relatively high thermal conductivity, of approximately 100-200 Watts per metre-Kelvin. In a sintered form, aluminium nitride and silicon carbide can have a thermal conductivity of less than 100 Watts per metre-Kelvin. The porous ceramic body may have any thickness. The thickness of the porous ceramic body may refer to the extension of the porous ceramic body in a direction between the liquid absorption surface and the heating surface. This may correspond to the direction of the liquid flow path through the porous ceramic body. The porous ceramic body may have a thickness which depends on the thermal properties of the material it is made from and the liquid it contains. The porous ceramic body may have a thickness of at least 1 millimetre. For example, the porous ceramic body may have a thickness of at least 2 millimetres, at least 3 millimetres, at least 4 millimetres, or at least 5 millimetres.

The porous ceramic body may have a thickness of no more than 10 millimetres. For example, the porous ceramic body may have a thickness of no more than 9 millimetres, no more than 8 millimetres, no more than 7 millimetres, or no more than 6 millimetres.

The porous ceramic body may have a thickness of between 1 millimetre and 10 millimetres. For example, the porous ceramic body may have a thickness of between 2 millimetres and 9 millimetres, between 3 millimetres and 8 millimetres, between 4 millimetres and 7 millimetres, or between 5 millimetres and 6 millimetres. The porous ceramic body may have a thickness of about 5 millimetres.

The heating element may have any thickness. The thickness of the heating element may refer to the extension of the heating element in a direction between the liquid absorption surface and the heating surface. This may correspond to the direction of the liquid flow path through the porous ceramic body. The heating element may have a thickness of at least 1 micrometre. The heating element may have a thickness of at least 2 micrometres. The heating element may have a thickness of at least 5 micrometres. The heating element may have a thickness of at least 200 micrometres. The heating element may have a thickness of at least 220 micrometres.

The heating element may have a thickness of less than 300 micrometres. The heating element may have a thickness of less than 250 micrometres. The heating element may have a thickness of less than 50 micrometres. The heating element may have a thickness of less than 20 micrometres.

The heating element may have a thickness of between 1 millimetre and 10 millimetres. The heating element may have a thickness of between 1 millimetre and 5 millimetres. The heating element may have a thickness of between 2 millimetres and 5 millimetres.

The heating element may have a thickness of between 200 micrometre and 300 micrometres. The heating element may have a thickness of between 200 micrometres and 250 micrometres. The heating element may have a thickness of between 220 micrometres and 300 micrometres.

According to an example of the present disclosure, there is provided a cartridge. The cartridge may comprise a heater assembly. The cartridge may comprise a liquid storage portion for holding an aerosol-forming substrate. The heater assembly may comprise a heating element for vaporising the liquid aerosol-forming substrate. The heater assembly may comprise a porous ceramic body for conveying the liquid aerosol-forming substrate to the heating element. The porous ceramic body may have a liquid absorption surface and a heating surface. The heating element may be located on and bonded to the heating surface of the porous ceramic body.

According to an example of the present disclosure, there is provided a cartridge, the cartridge comprising a heater assembly and a liquid storage portion for holding a liquid aerosol-forming substrate, the heater assembly comprising: a heating element for vaporising the liquid aerosol-forming substrate; a porous ceramic body for conveying the liquid aerosol-forming substrate to the heating element. The porous ceramic body has a liquid absorption surface and a heating surface. The heating element is located on and bonded to the heating surface of the porous ceramic body.

The cartridge may comprise the liquid aerosol-forming substrate in the liquid storage portion. The liquid aerosol-forming substrate may be as described above.

The porous body may be fluidly connected to the liquid storage portion. The porous body may have a liquid absorption surface. The liquid absorption surface of the porous ceramic body may be fluidly connected to the liquid storage portion.

The liquid storage portion may be arranged at the liquid absorption surface of the porous ceramic body.

There is provided an aerosol-generating system. The aerosol-generating system may comprise a cartridge and an aerosol-generating device. The cartridge may comprise a heater assembly. The cartridge may comprise a liquid storage portion for holding an aerosol-forming substrate. The heater assembly may comprise a heating element for vaporising the liquid aerosol-forming substrate. The heater assembly may comprise a porous ceramic body for conveying the liquid aerosol-forming substrate to the heating element. The porous ceramic body may have a liquid absorption surface and a heating surface. The heating element may be located on and bonded to the heating surface of the porous ceramic body.

The aerosol-generating device may comprise a power supply for supplying electrical power to the heating element. The aerosol-generating device may comprise control circuitry configured to control a supply of power from the power supply to the heating element.

There is provided an aerosol-generating system comprising: a cartridge and an aerosol-generating device, the cartridge comprising a heater assembly and a liquid storage portion for holding a liquid aerosol-forming substrate, the heater assembly comprising: a heating element for vaporising the liquid aerosol-forming substrate; a porous ceramic body for conveying the liquid aerosol-forming substrate to the heating element. The porous ceramic body has a liquid absorption surface and a heating surface. The heating element is located on and bonded to the heating surface of the porous ceramic body.

The aerosol-generating device may comprise a power supply for supplying electrical power to the heating element; and control circuitry configured to control a supply of power from the power supply to the heating element.

The cartridge may comprise the liquid aerosol-forming substrate in the liquid storage portion. The liquid aerosol-forming substrate may be as described above.

The aerosol-generating system may be portable. The aerosol-generating system may have a size comparable to a conventional cigar or cigarette.

The cartridge may be removably couplable to the aerosol-generating device.

The aerosol-forming substrate may be liquid at room temperature. The aerosol-forming substrate may comprise both liquid and solid components. The liquid aerosol-forming substrate may comprise nicotine. The nicotine containing liquid aerosol-forming substrate may be a nicotine salt matrix. The liquid aerosol-forming substrate may comprise plant-based material. The liquid aerosol-forming substrate may comprise tobacco. The liquid aerosol-forming substrate may comprise a tobacco-containing material containing volatile tobacco flavour compounds, which are released from the aerosol-forming substrate upon heating. The liquid aerosol-forming substrate may comprise homogenised tobacco material. The liquid aerosol-forming substrate may comprise a non-tobacco-containing material. The liquid aerosol-forming substrate may comprise homogenised plant-based material.

The liquid aerosol-forming substrate may comprise one or more aerosol-formers. An aerosol-former is any suitable known compound or mixture of compounds that, in use, facilitates formation of a dense and stable aerosol and that is substantially resistant to thermal degradation at the temperature of operation of the system. Examples of suitable aerosol formers include glycerine and propylene glycol. Suitable aerosol-formers are well known in the art and include, but are not limited to: polyhydric alcohols, such as triethylene glycol, 1,3-butanediol and glycerine; esters of polyhydric alcohols, such as glycerol mono-, di- or triacetate; and aliphatic esters of mono-, di- or polycarboxylic acids, such as dimethyl dodecanedioate and dimethyl tetradecanedioate. The liquid aerosol-forming substrate may comprise water, solvents, ethanol, plant extracts and natural or artificial flavours.

The liquid aerosol-forming substrate may comprise nicotine and at least one aerosol-former. The aerosol-former may be glycerine or propylene glycol. The aerosol former may comprise both glycerine and propylene glycol. The liquid aerosol-forming substrate may have a nicotine concentration of between about 0.5% and about 10%, for example about 2%.

The airflow pathway may pass through the liquid storage portion. For example, the liquid storage portion may have an annular cross-section defining an internal passage or aerosol channel, and the airflow pathway may extend through the internal passage or aerosol channel of the liquid storage portion.

The cartridge may comprise a cartridge housing. The cartridge housing may be formed from a durable material. The cartridge housing may be formed from a liquid impermeable material. The cartridge housing may be formed form a mouldable plastics material, such as polypropylene (PP) or polyethylene terephthalate (PET) or a copolymer such as Tritan™, which is made from three monomers: dimethyl terephthalate (DMT), cyclohexanedimethanol (CHDM), and 2,2,4,4-tetramethyl-1,3-cyclobutanediol (CBDO). The cartridge housing may define a portion of the liquid storage portion or reservoir. The cartridge housing may define the liquid storage portion. The cartridge housing and the liquid storage portion may be integrally formed. Alternatively, the liquid storage portion may be formed separately from the outer housing and arranged in the outer housing.

The aerosol-generating device may comprise a power supply for supplying power to the heater assembly. The aerosol-generating device may comprise control circuitry for controlling the supply of power from the power supply to the heater assembly. The cartridge may be removably couplable to the aerosol-generating device.

The aerosol-generating device may comprise a housing. The housing may be elongate. The housing may comprise any suitable material or combination of materials. Examples of suitable materials include metals, alloys, plastics or composite materials containing one or more of those materials, or thermoplastics that are suitable for food or pharmaceutical applications, for example polypropylene, polyetheretherketone (PEEK) and polyethylene. The material is preferably light and non-brittle.

The aerosol-generating device housing may define a cavity for receiving a portion of a cartridge. The aerosol-generating device may have a connection end configured to connect the aerosol-generating device to a cartridge. The connection end may comprise the cavity for receiving the cartridge.

The power supply may be any suitable power supply. Preferably, the power supply is a DC power supply. The power supply may be a battery. The battery may be a Lithium based battery, for example a Lithium-Cobalt, a Lithium-Iron-Phosphate, a Lithium Titanate or a Lithium-Polymer battery. The battery may be a Nickel-metal hydride battery or a Nickel cadmium battery. The power supply may be another form of charge storage device such as a capacitor. The power supply may be rechargeable and be configured for many cycles of charge and discharge. The power supply may have a capacity that allows for the storage of enough energy for one or more user experiences of the aerosol-generating system; for example, the power supply may have sufficient capacity to allow for the continuous generation of aerosol for a period of around six minutes, corresponding to the typical time taken to smoke a conventional cigarette, or for a period that is a multiple of six minutes. In another example, the power supply may have sufficient capacity to allow for a predetermined number of puffs or discrete activations of the aerosol-generating system.

The control circuitry may comprise any suitable controller or electrical components. The controller may comprise a memory. Information for performing a method of operation of the device or system may be stored in the memory. The control circuitry may comprise a microprocessor. The microprocessor may be a programmable microprocessor, a microcontroller, or an application specific integrated chip (ASIC) or other electronic circuitry capable of providing control. The control circuitry may be configured to supply power to the heating element continuously following activation of the device, or may be configured to supply power intermittently, such as on a puff-by-puff basis. The power may be supplied to the heating element in the form of pulses of electrical current, for example, by means of pulse width modulation (PWM).

Features described in relation to one of the above examples may equally be applied to other examples of the present disclosure.

The heating element may be in contact with the heating surface of the porous ceramic body, and the heating element and the heating surface of the porous ceramic body are bonded by virtue of this contact. The heating element may be in contact with the entire heating surface of the porous ceramic body. Alternatively, the heating element may be in contact with only a portion of the heating surface of the porous ceramic body. The heating element may be in direct contact with the heating surface of the porous ceramic body. Alternatively, the heating element may be in indirect contact with the surface of the porous ceramic body. The contact between the heating element and the heating surface of the porous ceramic body may further improve the robustness and reliability of the connection between the heating element and the porous ceramic body. This may advantageously help to improve the transfer of heat between the heating element and the porous ceramic body. This also helps to prevent unwanted loss of heat, which helps to improve energy efficiency also helps with the generation of a more consistent aerosol. This further provides a user of the aerosol-generating system with an improved and more enjoyable experience. Such an arrangement may also help to reduce the likelihood of a user experiencing dry heating or a dry puff.

The heating element and the heating surface of the porous ceramic body may be bonded by an intervening layer located between the heating element and the heating surface of the porous ceramic body. The intervening layer may cover the entire heating surface of the porous ceramic body. Alternatively, the intervening layer may cover only a portion of the heating surface of the porous ceramic body. The intervening layer may be an adhesive. The intervening layer may be in direct contact with one or both of the heating surface of the porous ceramic body and the heating element. The intervening layer may advantageously help to more securely bond the heating element to the heating surface of the porous ceramic body. The intervening layer may also provide advantageous heat transfer properties between the heating element and the surface of the porous ceramic body. This may further improve the consistency of the aerosol produced and further improve the experience of a user of the aerosol-generating system.

The heating element may be a porous ceramic heating element. The heating element may be porous across the entire surface of the heating element. Alternatively, the heating element may not be porous across the entire surface of the heating element. For example, a first portion of the heating element may be porous and a second portion of the heating element may be non-porous. Embodiments in which the heating element is a porous ceramic heating element may be advantageous in that the aerosol-generating liquid may flow from the porous ceramic body into the heating element. This may improve energy efficiency of aerosol-generation and may also help with the generation of a more consistent aerosol.

The heating element may be a doped ceramic material. The heating element may be doped such that the heating element is electrically conductive. Doping the ceramic material may be advantageous in that it avoids altering the porosity of ceramic material when it is a porous ceramic material. This is can be preferable to other known techniques of forming a heating element, which involve depositing the heating element by thin film or thick film techniques, which can reduce the properties of the ceramic material, in particular the porosity. The thickness of the doped portion may be increased where the cross sectional area of the heating portion is smaller or where the heating resistance required is higher. The dopant used to dope the ceramic heating member may be an n-type dopant or a p-type dopant. The dopant may be any one of, but not limited to, nitrogen, phosphorous, aluminium or boron. The interface between the heating portion and the porous portion may comprise a portion of partially doped ceramic material. In other words, an end of the interface adjacent the heating portion may be doped to substantially the same extent as the heating portion and an end of the interface adjacent the porous portion may be substantially undoped.

The ceramic material may be doped by ion implantation. Ion implantation involves the implantation of ions into a layer of bulk material or the exchange of ions, taking one species out and replacing it by another. Ion implantation can be carried out chemically or physically.

The ceramic material may be doped by transmutation. Transmutation changes one species of atom already present in the material into another by irradiation with particles, such as neutrons or alpha particles, which leads to a short lived decay process leading to a stable isotope which was not present in the original material. Transmutation is advantageous in that the doping occurs directly in the ceramic material and does not require the bonding or attachment of an additional electrically conductive material. Transmutation therefore provides a more monolithic approach.

The heating element may be a metallic heating element, such as a metallic heating track. This may be advantageous in that the heating element may be pre-formed and the porous ceramic body may be formed and bonded simultaneously to the heating element. This may also help to simplify the manufacturing of the heater assembly by reducing manufacturing times and providing a more cost effective solution. This advantageously creates a tight mechanical connection between the metallic heating element and the porous ceramic body as the metallic heating element may be inside assembled fully or partially inside the porous ceramic body.

The heating element may be deposited on the porous ceramic body by thick film techniques such as screen printing, inkjet printing, aerosol-jet printing or laser direct structuring. Such printing processes may be particularly advantageous when used in high speed production processes.

The heating element may be deposited by thin film techniques such as physical vapor deposition or chemical vapor deposition. This may provide several advantages, including more uniform heating for the heating element. For example, such techniques may allow for a wider variety in the design and layout of the heating element, without unduly compromising any desired resistivity targets for the heating element. Furthermore, such deposition techniques may result in the deposition of less material within the voids of the ceramic body itself, when compared to, for example, thicker film deposition techniques. The design of pores or gaps in the heating element may therefore be more closely correlated to that of the ceramic body.

The heating element and the porous ceramic body may be bonded by molding. For example, the heating element may be pre-formed and the porous ceramic body may be molded around the pre-formed heating element. This may also help to simplify the manufacturing of the heater assembly by reducing manufacturing times and providing a more cost effective solution. This advantageously creates a tight mechanical connection between the heating element and the porous ceramic body as the heating element may be inside assembled fully or partially inside the porous ceramic body.

The heating element and the porous ceramic body may be formed separately and assembled to form the heater assembly. This may advantageously allow for increased manufacturing freedom as the two components can be manufactured by separate techniques or in separate locations prior to being assembled. Consequently, this can also help to reduce the need for specialised equipment at the assembly location, thus reducing the constraints and cost associated with the manufacturing of the heating element.

The liquid absorption surface of the porous ceramic body may have an area that is different to an area of the heating surface of the porous ceramic body. The porous ceramic body may be substantially incompressible. The porous ceramic body may be incompressible.

A heater assembly having a heating surface with the same area as the liquid absorption surface may be inefficient due to heat generated by the heater not being used to vaporise an aerosol-forming substrate. An inefficient heater assembly provides a reduced throughput of aerosol.

Advantageously, providing a porous ceramic body in which the heating surface and the liquid absorption surface have different areas may improve the throughput of aerosol that can be generated by the heater assembly compared to a heater assembly in which the heating surface has the same area as the liquid absorption surface.

Increasing heating efficiency may reduce power consumption during use of the heater assembly.

The area of the heating surface of the porous ceramic body may be less than the area of the liquid absorption surface of the porous ceramic body. The area of the liquid absorption surface of the porous ceramic body may be greater than the area of the heating surface of the porous ceramic body.

Advantageously, when the porous ceramic body has a shape such that the heating surface has a smaller area than the liquid absorption surface, heat flow from the heating element towards the liquid absorption surface and then to the liquid storage portion by conduction may be reduced. The relatively smaller heating surface provides a small heat transfer area through which heat can be transferred, by conduction, from the heating element to the porous ceramic body, and towards the liquid absorption surface.

Decreasing heat loss from the heating element to the bulk of the porous ceramic body may consequently increase heating efficiency because more of the heat energy provided by the heating element may be used to vaporise the aerosol-forming substrate. Consequently, the porous ceramic body having a shape such that the heating surface has a smaller area than the liquid absorption surface may increase the throughput of aerosol generated by the heater assembly.

Advantageously, the porous ceramic body having a shape such that the heating surface has a smaller area than the liquid absorption surface may reduce the area of the heating surface that is not close enough to the heating element to allow aerosol-forming substrate being conveyed to the heating surface to be vaporised. In other words, the size and shape of the heating surface may more closely match with the size and shape of the heating element. Consequently, more of the liquid aerosol-forming substrate may be conveyed from the liquid absorption surface to an area of the heating surface that is near to the heating element, which may result in more of the liquid aerosol-forming substrate at the heating surface being vaporised. More liquid aerosol-forming substrate being vaporised may increase the throughput of aerosol generated by the heater assembly. Further, this arrangement may allow for the power density at the heating surface to be maximised, which also improves heating efficiency.

Advantageously, the liquid absorption surface having a larger area than the heating surface may allow the liquid absorption surface to receive a larger volume of liquid aerosol-substrate from a liquid storage portion. As a consequence of the relatively smaller area of the heating surface, as the liquid aerosol-forming substrate is conveyed through the porous ceramic body and towards the heating surface, the flow rate of the liquid aerosol-forming substrate to the heating element may be higher than with a typical heater assembly. A higher flow rate of liquid aerosol-forming substrate at the heating element may increase the throughput of aerosol generated by the heater assembly.

The area of the heating surface of the porous body may be greater than the area of the liquid absorption surface of the ceramic porous body. The area of the liquid absorption surface of the porous body may be less than the area of the heating surface of the porous ceramic body.

Advantageously, when the porous ceramic body has a shape such that the liquid absorption surface has a smaller area than the heating surface, the smaller area of the liquid absorption surface may cause a reduction in heat flow through the aerosol-forming substrate from the heating element to the liquid absorption surface via heat conduction. Reducing heat flow from the heating surface to the liquid absorption surface may consequently increase thermal efficiency because more of the heat energy provided by the heating element may be used to vaporise the liquid aerosol-forming substrate. Consequently, the porous ceramic body having a shape such that the liquid absorption surface has a smaller area than the heating surface may provide for increased heating efficiency, which may increase the throughput of aerosol generated by the heater assembly.

Advantageously, the porous ceramic body having a shape such that the liquid absorption surface has a smaller area than the heating surface may reduce the area of the heating surface that is not close enough to the heating element to allow aerosol-forming substrate being conveyed to the heating surface to be vaporised. In other words, the size and shape of the heating surface may more closely match with the size and shape of the heating element. Consequently, more of the liquid aerosol-forming substrate being may be conveyed from the liquid absorption surface and to an area of the heating surface that is near to the heating element, which may result in more of the liquid aerosol-forming substrate at the heating surface being vaporised. More liquid aerosol-forming substrate being vaporised may increase the throughput of aerosol generated by the heater assembly.

The heating surface of the porous ceramic body may be convex in one or both of a first transverse direction and a second transverse direction, the first transverse direction being orthogonal to the second transverse direction.

Inclusion of such a porous ceramic body may enable the surface area of the heating surface to be increased without increasing a width of the heating surface. This may increase the efficiency of the aerosol-generating system at vaporising liquid aerosol-forming substrate, whilst helping to avoid the need to redesign other components of the aerosol-generating system to accommodate the porous ceramic body.

The provision of a heating surface that is convex along one or both of a first transverse direction and a second transverse direction may help to avoid or minimise recirculation of airflow adjacent the heater assembly. In particular, a heating surface that is convex may help to avoid or minimise recirculation of airflow adjacent to a central region of the heater assembly. This may reduce a level of turbulence in the airflow adjacent to the heater assembly. Reducing a level of turbulence in the airflow adjacent to the heater assembly may improve the entrainment of vapour of aerosol-forming substrate in the airflow. This may improve the quality of the aerosol generated by the aerosol-generating system.

Improving the entrainment of vapour in the airflow through the aerosol-generating system may avoid or reduce vapour condensing to form large droplets of liquid aerosol-forming substrate. This may help to avoid an unpleasant and undesirable user experience.

Improving the entrainment of vapour in the airflow through the aerosol-generating system may avoid or reduce vapour condensing on internal surfaces of the aerosol-generating system. This may help to avoid or minimise damage to the aerosol-generating system and may allow optimal function of the aerosol-generating system.

The heating surface of the porous ceramic body may be convex in a single transverse direction.

The heating surface of the porous ceramic body may be convex in both the first transverse direction and the second transverse direction.

The heating surface of the porous ceramic body may be convex in one or both of the first transverse direction and the second transverse direction based on the configuration of the heater assembly relative to one or more airflow pathways of the aerosol-generating system. The heater assembly may be configured to minimise a level of turbulence in the airflow adjacent to the heater assembly. For example, it may be advantageous for the heater assembly to be arranged in the aerosol-generating system such that air drawn into the aerosol-generating system follows a curved path along at least a portion of a curved surface of the heater assembly.

The heating element may be convex in one or both of the first transverse direction and the second transverse direction.

The curvature of the heating element in the first transverse direction may be substantially the same as the curvature of the heating surface of the porous ceramic body in the first transverse direction. The curvature of the heating element in the second transverse direction may be substantially the same as the curvature of the heating surface of the porous body in the second transverse direction. The curvature of the heating element in both the first transverse direction and the second transverse direction may be substantially the same as the curvature of the heating surface of the porous body in both the first transverse direction and the second transverse direction, respectively.

The average pore size of the porous ceramic body may vary between the liquid absorption surface and the heating surface.

The porous ceramic body may include a first average pore size at the liquid absorption surface, and a second average pore size at the heating end. The first average pore size may be greater than the second average pore size.

The first pore size at the liquid absorption surface may be about 150 micrometres. The second pore size at the heating end may be about 20 micrometres. The pore size may vary linearly between the first pore size and the second pore size to provide a pore size gradient between the liquid absorption surface and the heating end of the porous ceramic body.

The pore structure and pore size gradient in the porous ceramic body may be achieved by etching the pores into a portion of silicon carbide.

The heater assembly may comprise a thermally insulating layer. The thermally insulating layer may have a lower thermal conductivity than the porous ceramic body. The thermally insulating layer may be disposed between each of the porous ceramic body and the heating element. The thermally insulating layer may be in direct contact with the heating element. Alternatively, the thermally insulating layer may be in indirect contact with the heating element. The thermally insulating layer may be in direct contact with the heating surface of the porous ceramic body. Alternatively, the thermally insulating layer may be in indirect contact with the heating surface of the porous ceramic body. The thermally insulating layer may be in direct contact with each of the heating surface porous ceramic body and the heating element. Alternatively, the thermally insulating layer may be in indirect contact with each of the heating surface porous ceramic body and the heating element. The thermally insulating layer may be in direct contact with one or bth The thermally insulating layer may be configured to reduce heat transfer from the heating element to the porous ceramic body.

With this arrangement, the heat losses from the heating element to the porous ceramic body, and to liquid within the porous ceramic body, are reduced. This provides a more efficient heater assembly in which the amount of use and number of uses of the device by a user can be increased, before the device power supply, such as a battery, is depleted. The inventors have estimated that in a known device, approximately one third of energy from the heating element is lost through conduction in the porous body and liquid in the porous body. The remaining two thirds are used to generate an aerosol by heating a liquid aerosol-forming substrate. In the arrangement described herein, these energy losses are reduced. Specifically, the thermally insulting layer reduces heat propagation or conduction from the heating element towards or through the porous ceramic body. This reduction in conduction can concentrate heat to the heating surface of the porous ceramic body, minimising heat dissipation and increasing heating efficiency of the heater assembly.

The thermally insulating layer may comprise a thermally insulating material. The thermally insulating material may have a lower thermal conductivity than the porous ceramic body. The thermally insulating material may have a higher porosity than the porous ceramic body. This has the advantage of providing a thermally insulating layer which is particularly effective at reducing energy losses, while being easy to manufacture.

The thermally insulating layer may comprise a material having a thermal conductivity of less than 40 Watts per metre-Kelvin. This has the advantage of providing a thermally insulating layer which is effective at reducing energy losses through the porous ceramic body. The thermally insulating layer may comprise a material having a thermal conductivity of less than 10 Watts per metre-Kelvin. This has the advantage of providing a thermally insulating layer which is particularly effective at reducing energy losses through the porous ceramic body.

The thermally insulating layer may extend entirely between the porous ceramic body and the heating element. This has the advantage of more effectively providing a barrier between the heating element and the porous ceramic body, and as such is particularly effective at reducing energy losses through the porous ceramic body.

The thermally insulating layer may comprise one or more of: alumina, zirconia, zirconia with magnesium oxide, glass ceramic, quartz, a porous polymer. The porous polymer may be polyimide.

The thermally insulating layer may comprise alumina having a thermal conductivity of 20-40 Watts per metre-Kelvin. The thermally insulating layer may comprise a material having a thermal conductivity of less than 10 Watts per metre-Kelvin, such as zirconia with or without magnesium oxide, glass ceramics, quartz. Use of alumina, zirconia with or without magnesium oxide, glass ceramics, quartz, is advantageous, as these materials are compatible with a manufacturing process involving sintering, and as such a heater assembly having a thermally insulating layer of one of these materials is more easily manufactured.

The thermally insulating layer may have a thickness of between 0.1 mm and 2 mm. A thermally insulating layer with such a thickness is particularly suited to reducing energy losses from the heating element to the porous ceramic body. Preferably, the thermally insulating layer has a thickness of between 0.5 mm and 1.5 mm. A thermally insulating layer with such a thickness is further suited to reducing energy losses from the heating element to the porous ceramic body.

The heating element may comprise a plurality of tracks or track portions arranged electrically in parallel. The heating element resistance at room temperature may be between 0.5 Ohms and 1.5 Ohms, preferably between 0.7 Ohms and 1.3 Ohms, and more preferably 1 Ohm. The resistance of the heating element may be matched to requirements of control electronics.

At least two of the electrically parallel heating tracks may have similar resistances to each other, or have the same resistance as each other. Preferably, all of the electrically parallel heating tracks are of similar or of the same resistance as each other. The heating tracks arranged electrically in parallel may have different resistances, which is particularly beneficial in a heater assembly where it is advantageous for zones of the heating element to generate different power levels. This could be the case, for example, to compensate for higher thermal losses in an outer part of the heating element. As such, heating tracks on an exterior or outer part of the heating element may be designed to have a lower resistance (which can generate more heat) than heating tracks in the centre of the heating element.

The heating element may comprise a plurality of tracks or track portions. The plurality of tracks or track portions may be arranged electrically in parallel. By being arranged electrically in parallel, current flow is split into separate parallel flow paths, the separate parallel flow paths being subsequently re-combined.

The heating element may comprise a first connecting pad and a second connecting pad. The first or second connecting pads (or first and second connecting pads) may be configured to allow connection to an external circuit. An aperture or plurality of apertures in the heating element may separate each track or track portion. The heating element may comprise at least one diverging portion, in which current is split from the first connecting pad into track portions. The track portions define electrically parallel paths. The heating element may comprise a converging portion. In the converging portion, current is combined from track portions which define electrically parallel paths, into the second connecting pad.

Various different arrangements are possible of tracks or track portions arranged electrically in parallel. The heating element may comprise two, three, four or more track portions which define electrically parallel paths.

By having tracks or track portions arranged electrically in parallel, if one track portion is defective, current can be redistributed and can still flow through the heating element, i.e. the electrical connection between the first connecting pad and the second connecting pad is not broken. In contrast, in a simple serpentine heater defining a single electrical path between the first connecting pad and the second connecting pad, if a part of the serpentine heating element is broken or contains a defect, this can cause an increase in local resistance, causing increased power dissipation, which in turn increases the resistance until breakage.

The inventors have also identified that the electrically parallel tracks or track portions have a surprising additional advantage. In such an arrangement, in case of breakage of one track portion, the heating element will still operate and can, for an initial transitory period, operate in an advantageous way because the breakage of one track or track portion would result in a higher energy density on the remaining tracks or track portions. In such a case, the same power would still be provided but over a smaller area, so throughput of the aerosol-generating substrate is increased. Such a breakage causing an increase in current on unbroken tracks or track portions can eventually affect the user's experience. This can be mitigated for by a mechanism to alert the user about possible future below optimal performance of the heater assembly. Electrically parallel tracks have the advantage of increasing the number of puffs before full failure of the heater, and potentially increasing the heater lifetime up to the lifetime of the device.

The heating element may comprise a plurality of tracks or track portions defining a path having at least one bend, the inner edge of the bend being curved.

The inner edge of the bend being curved has the advantage of guiding current to flow in a more evenly distributed way around the at least one bend. This reduces a current concentration which in turn limits hot spot creation.

The heating element may comprise a plurality of tracks or track portions having a gradient of electrical resistivity perpendicular to current flow in a corner or corners, such that the electrical resistivity is higher at an inner part of the corner and lower at an outer part of the corner. Such a gradient is beneficial to counterbalance localized high current density and reduce hot spot creation.

The present disclosure also relates to a method of manufacturing a heater assembly for an aerosol-generating system. The method may comprise forming a heating element for vaporising a liquid aerosol-forming substrate. The method may comprise forming a porous ceramic body for conveying the liquid aerosol-forming substrate to the heating element, the porous ceramic body having a liquid absorption surface and a heating surface. The method may comprise bonding the heating element on the heating surface of the porous ceramic body.

According to the present invention, there is also provided a method of manufacturing a heater assembly for an aerosol-generating system. The method comprises forming a heating element for vaporising a liquid aerosol-forming substrate. The method comprises forming a porous ceramic body for conveying the liquid aerosol-forming substrate to the heating element, the porous ceramic body having a liquid absorption surface and a heating surface. The method comprises bonding the heating element on the heating surface of the porous ceramic body.

The method may comprise a step of adding a template to the heating element material. The template may be removable once the heating element is formed. This may advantageously allow a porous structure to be created in the heating element to enhance aerosol-generating liquid vaporisation.

According to an example of the present disclosure, there is provided an aerosol-generating system. The aerosol-generating system may comprise a heater assembly as discussed above. The heating element may be fluid permeable such that, in use, vapour is emitted from the heater assembly in an average vapour emission direction. The aerosol-generating system may further comprise an air inlet and an aerosol outlet. The air inlet may be in fluid communication with the aerosol outlet to define an airflow pathway through the aerosol-generating system. The heater assembly may be arranged in fluid communication with the airflow pathway such that air flows past the heater assembly in an average airflow direction. The heater assembly and airflow pathway may be arranged such that an angle between the average vapour emission direction and the average airflow direction is less than 135 degrees.

Advantageously, by arranging the heater assembly and airflow pathway such that the angle between the average vapour emission direction and the average airflow direction is less than 135 degrees, the average airflow direction does not directly oppose the average vapour emission direction. Therefore, the momentum of the vapour and the airflow is not reduced to the same extent as when the average airflow direction does directly oppose the average vapour emission direction. This reduces the tendency for recirculation and turbulence to occur in the airflow path and the vapour is less likely to impinge on the internal surfaces of the aerosol-generating system. Accordingly, condensation of aerosol within the aerosol-generating system is less likely to occur.

The average vapour emission direction may be substantially perpendicular to the heating surface of the porous ceramic body. As used herein, the term “substantially perpendicular” means 90 degrees plus or minus 10 degrees, preferably plus or minus 5 degrees.

An advantage of the average vapour emission direction being substantially perpendicular to the heating surface of the porous ceramic body is that it makes orientating the average vapour emission direction relative to the average airflow direction straightforward because the vapour will be emitted substantially perpendicular to the heating surface of the of the porous ceramic body. Therefore, by angling the heater assembly appropriately relative to the airflow in the airflow pathway or vice versa, a desired angle between the average vapour emission direction and average airflow direction can be achieved.

The heater assembly and airflow pathway may be arranged such that an angle between the average vapour emission direction and the average airflow direction is less than 110 degrees, preferably less than 100 degrees.

The heater assembly and airflow pathway may be arranged such that an angle between the average vapour emission direction and the average airflow direction is approximately 90 degrees. This arrangement results in the vapour being emitted at an angle substantially perpendicular to the average airflow direction. The average vapour emission direction has no speed or direction component that opposes the airflow direction and therefore any loss of momentum of the airflow is reduced. This reduces the tendency for recirculation and turbulence to occur in the airflow path and the vapour is less likely to impinge on the internal surfaces of the aerosol-generating system. Furthermore, entrainment of the vapour in the airflow is improved. Accordingly, condensation of aerosol within the aerosol-generating system is less likely to occur.

The heater assembly and airflow pathway may be arranged such that an angle between the average vapour emission direction and the average airflow direction is less than 90 degrees. In this arrangement, the average vapour emission direction has no speed or direction component that opposes the airflow direction and actually has a speed and direction component in the same direction as the average airflow direction. Therefore, any loss of momentum of the airflow is further reduced. This reduces the tendency for recirculation and turbulence to occur in the airflow path and the vapour is less likely to impinge on the internal surfaces of the aerosol-generating system. Furthermore, entrainment of the vapour in the airflow is improved. Accordingly, condensation of aerosol within the aerosol-generating system is less likely to occur.

The heater assembly and airflow pathway may be arranged such that an angle between the average vapour emission direction and the average airflow direction is approximately 45 degrees. The heater assembly and airflow pathway may be arranged such that an angle between the average vapour emission direction and the average airflow direction is less than 45 degrees.

The heater assembly and airflow pathway may be arranged such that the average vapour emission direction and the average airflow direction are substantially the same. In this arrangement, there is virtually no loss of momentum of the airflow as the average vapour emission direction and average airflow direction are the same. This reduces the tendency for recirculation and turbulence to occur in the airflow path and the vapour is less likely to impinge on the internal surfaces of the aerosol-generating system. Furthermore, entrainment of the vapour in the airflow is improved. Accordingly, condensation of aerosol within the aerosol-generating system is less likely to occur.

A cross-sectional area of the airflow pathway in the region of the heater assembly may be configured such that, in use, the airflow speed is between 0.1 and 2 metres per second, preferably between 0.5 and 1.5 metres per second and more preferably approximately 1 metre per second. This range of airflow speeds has been found to effectively entrain the vapour emitted from different designs of heating element without excessively cooling the heating element.

The heating element may comprise a porous layer of electrically conductive material. Advantageously, a heating element comprising a porous layer of electrically conductive material allows an electrical current to flow through the heating element such that the heating element can be resistively heated and also allows vapours to travel through the heating element via the pores in its porous structure. Thus vapour emission occurs through the porous heating element. This avoids the build-up of vapour pressure underneath the heating element and high speed vapour emission at the sides of the heating element. The inventors have found that this arrangement produces a consistent vapour across the heating element and a lower vapour emission speed of approximately 0.1 metres per second. Such a low vapour emission speed means that the vapour is easily carried away by the airflow reducing the impingement of vapour on the internal walls of the aerosol-generating system.

Features described above in relation to the heating element of the present disclosure and their associated advantages may equally be applied to the method of the present disclosure.

Below, there is provided a non-exhaustive list of non-limiting examples. Any one or more of the features of these examples may be combined with any one or more features of another example, embodiment, or aspect described herein.
EX1. A heater assembly for an aerosol-generating system, the heater assembly comprising: a heating element for vaporising a liquid aerosol-forming substrate; and a porous ceramic body for conveying the liquid aerosol-forming substrate to the heating element, the porous ceramic body having a liquid absorption surface and a heating surface, the heating element being located on and bonded to the heating surface of the porous ceramic body.
EX2. A heater assembly according to EX1, wherein the heating element is in contact with the heating surface of the porous ceramic body, and the heating element and the heating surface of the porous ceramic body are bonded by virtue of this contact.
EX3. A heater assembly according to EX1, wherein the heating element and the heating surface of the porous ceramic body are bonded by an intervening layer located between the heating element and the heating surface of the porous ceramic body.
EX4. A heater assembly according to any one of EX1 to EX3, wherein the heating element is a porous ceramic heating element.
EX5. A heater assembly according to EX4, wherein the heating element is a doped portion of the porous ceramic heating element.
EX6. A heater assembly according to any one of EX1 to EX3, wherein the heating element is a metallic heating element, such as a metallic heating track.
EX7. A heater assembly according to any of EX1 to EX4, wherein the heating element is deposited on the porous ceramic body by thick film techniques such as screen printing, inkjet printing, aerosol-jet printing or laser direct structuring.
EX8. A heater assembly according to any one of EX1 to EX4, wherein the heating element is deposited by thin film techniques such as physical vapor deposition or chemical vapor deposition.
EX9. A heater assembly according to any one of EX1 to EX6, wherein the heating element and the porous ceramic body are bonded by molding.
EX10. A heater assembly according to any one of EX1 to EX6, wherein the heating element and the porous ceramic body are formed separately and assembled to form the heater assembly.
EX11. A heater assembly according to any one of EX1 to EX10, wherein the liquid absorption surface of the porous ceramic body has an area that is different to an area of the heating surface of the porous body.
EX12. A heater assembly according to any one of EX1 to EX11, wherein the heating surface of the porous body is convex in one or both of a first transverse direction and a second transverse direction, the first transverse direction being orthogonal to the second transverse direction.
EX13. A heater assembly according to any preceding claim, wherein the average pore size of the porous ceramic body varies between the liquid absorption surface and the heating surface.
EX14. A heater assembly according to any one of EX1 to EX13, wherein the heater assembly further comprises a thermally insulating layer having a lower thermal conductivity than the porous body, wherein the thermally insulating layer is disposed between and is in contact with each of the porous body and the heating element, and the thermally insulating layer is configured to reduce heat transfer from the heating element to the porous body.
EX15. The heater assembly according to any one of EX1 to EX15, wherein the heating element comprises a plurality of tracks or track portions arranged with a distance between at least two of the plurality of tracks or track portions in the range 150 to 300 micrometres.
EX16. A method of manufacturing a heater assembly for an aerosol-generating system, the method comprising: forming a heating element for vaporising a liquid aerosol-forming substrate; forming a porous ceramic body for conveying the liquid aerosol-forming substrate to the heating element, the porous ceramic body having a liquid absorption surface and a heating surface; bonding the heating element on the heating surface of the porous ceramic body.
EX17. A method of manufacturing a heater assembly for an aerosol-generating system according to EX16, wherein the step of bonding the heating element on the heating surface of the porous ceramic body comprises bonding the heating element in contact with the heating surface of the porous ceramic body, such that the heating element and the heating surface of the porous ceramic body are bonded by virtue of this contact.
EX18. A method of manufacturing a heater assembly for an aerosol-generating system according to EX16, wherein the step of bonding the heating element on the heating surface of the porous ceramic body comprises bonding the heating element and the heating surface of the porous ceramic body to an intervening layer between the heating element and the heating surface of the porous ceramic body.
EX19. A method of manufacturing a heater assembly for an aerosol-generating system according to any one of EX16 to EX18, wherein the heating element is a porous ceramic heating element
EX20. A method of manufacturing a heater assembly for an aerosol-generating system according to any one of EX16 to EX18, wherein the heating element is a metallic heating element, such as a metallic heating track.
EX21. A method of manufacturing a heater assembly for an aerosol-generating system according to any one of EX16 to EX18, wherein the step of bonding the heating element on the heating surface of the porous ceramic body comprises depositing the heating element on the porous ceramic body by thick film techniques such as screen printing, inkjet printing, aerosol-jet printing or laser direct structuring.
EX22. A method of manufacturing a heater assembly for an aerosol-generating system according to any one of EX16 to EX21, wherein the step of bonding the heating element on the heating surface of the porous ceramic body comprises depositing the heating element on the porous ceramic body by thin film techniques such as physical vapor deposition or chemical vapor deposition.
EX23. A method of manufacturing a heater assembly for an aerosol-generating system according to any one of EX16 to EX22, wherein the step of bonding the heating element on the heating surface of the porous ceramic body comprises molding the heating element and the porous ceramic body.
EX24. An aerosol-generating system comprising the heater assembly of any of EX1 to EX15, wherein the heating element is fluid permeable such that, in use, vapour is emitted from the heater assembly in an average vapour emission direction; wherein the aerosol-generating system further comprises an air inlet and an aerosol outlet, the air inlet being in fluid communication with the aerosol outlet to define an airflow pathway through the aerosol-generating system; wherein the heater assembly is arranged in fluid communication with the airflow pathway such that air flows past the heater assembly in an average airflow direction, wherein the heater assembly and airflow pathway are arranged such that an angle between the average vapour emission direction and the average airflow direction is less than 135 degrees.

The invention will now be further described, by way of example only, with reference to the accompanying drawings in which:

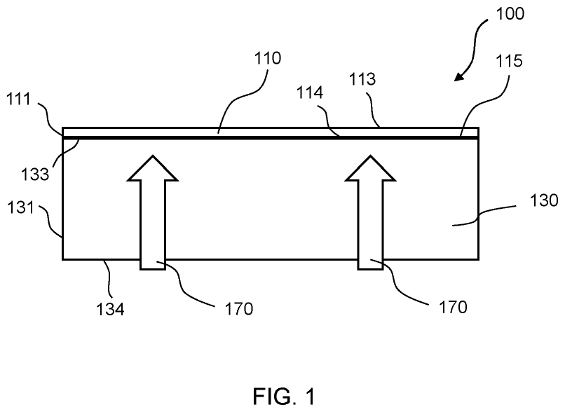

FIG. 1 shows a schematic illustration of a cross-section through a heater assembly in accordance with an example of the present disclosure, in which the heating element is a porous layer.

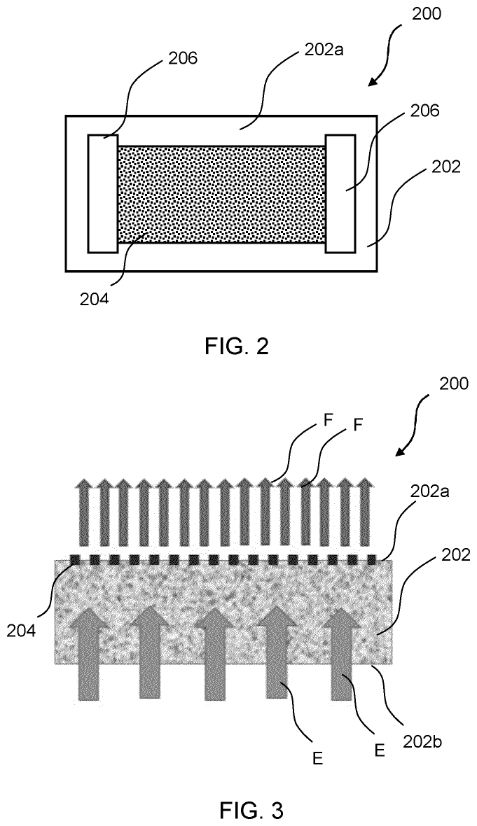

FIG. 2 shows a schematic illustration of a heater assembly in accordance with an example of the present disclosure.

FIG. 3 shows a schematic cross-sectional view of the heater assembly of FIG. 2.

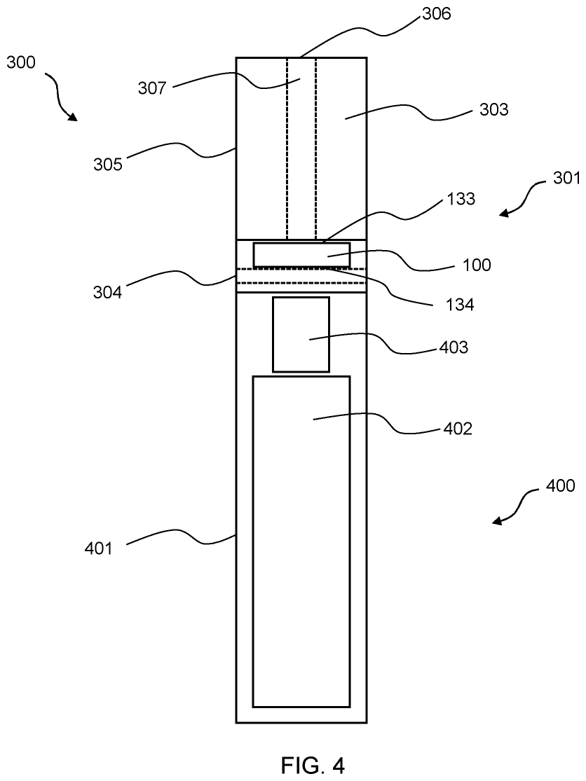

FIG. 4 is a schematic illustration of the interior of an aerosol-generating system according to an example of the present disclosure.

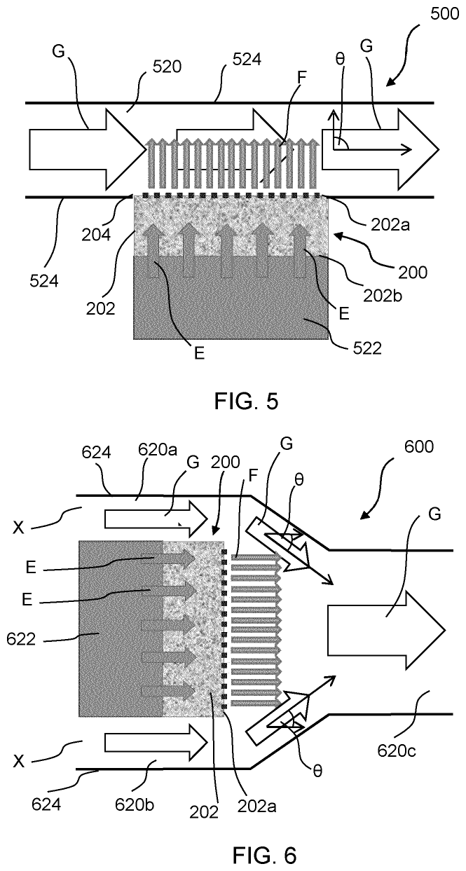

FIG. 5 is a schematic cross-sectional view of part of an aerosol-generating system according to another example of the present disclosure showing an arrangement of a heater assembly relative to an airflow pathway within the aerosol-generating system.

FIG. 6 is a schematic cross-sectional view of part of an aerosol-generating system according to another example of the present disclosure showing another arrangement of a heater assembly relative to an airflow pathway within the aerosol-generating system.

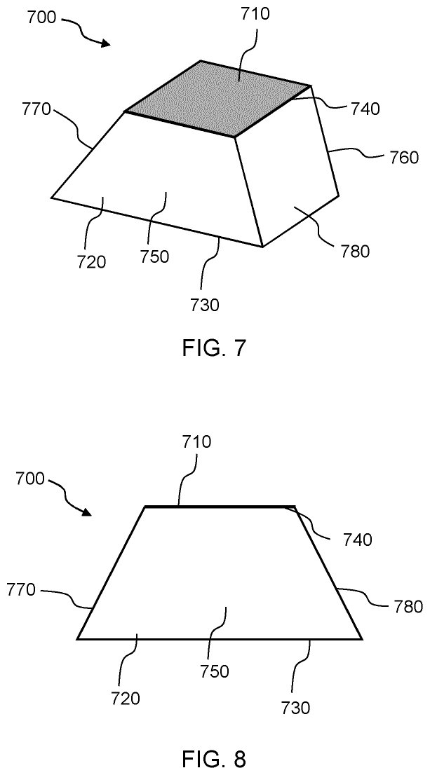

FIGS. 7 and 8 show schematic illustrations of an example of a heater assembly for an aerosol-generating system.

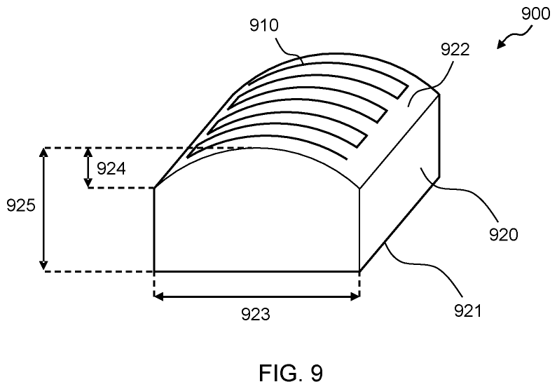

FIG. 9 shows a heater assembly for use in an aerosol-generating system according to an example of the present disclosure, the heater assembly comprises a heating element for vaporising a liquid aerosol-forming substrate.

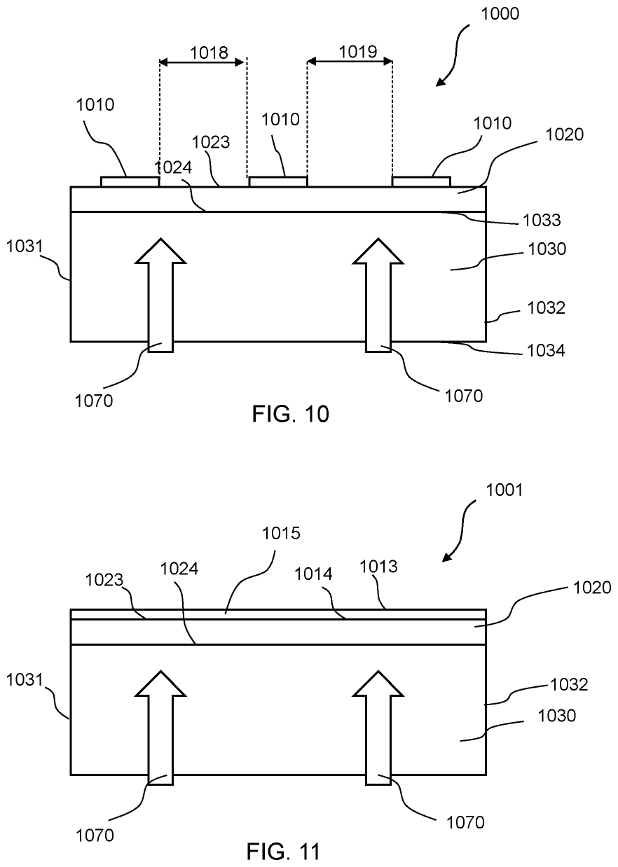

FIG. 10 shows a schematic cross-sectional view of a heater assembly for an aerosol-generating system according to an example of the present disclosure.

FIG. 11 shows a schematic cross-sectional view of another example heater assembly for an aerosol-generating system according to an example of the present disclosure.

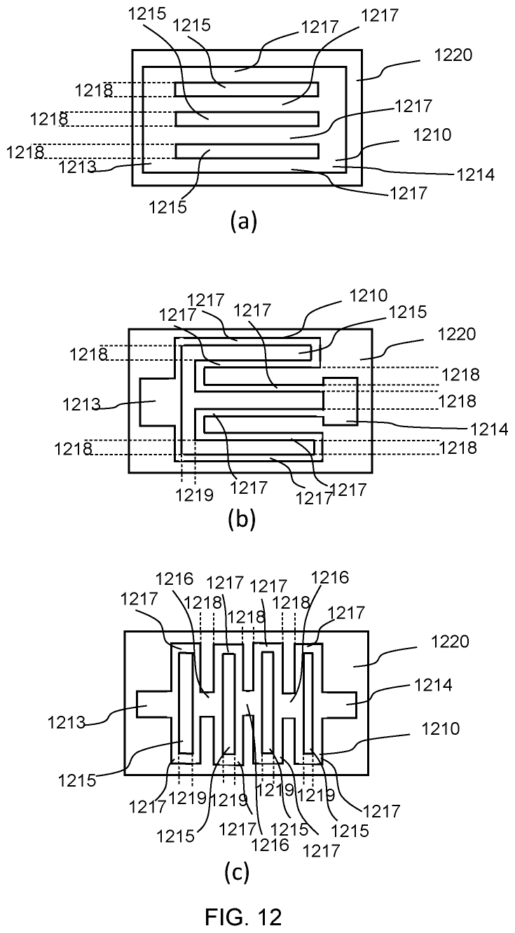

FIGS. 12 (a) to 12 (c) show schematic illustrations of different heating elements for an aerosol-generating system according to an example of the present disclosure.

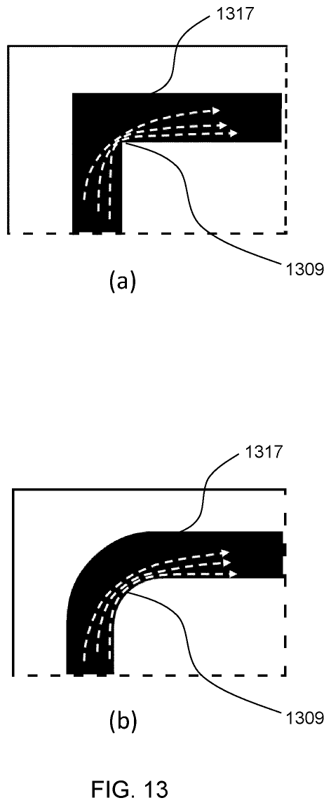

FIGS. 13 (a) and 13 (b) show schematic illustrations of current flow around a corner of a heating element track according to an example of the present disclosure.

Embodiments of the invention will now be described in detail, by way of example only, with reference to the accompanying drawings, in which:

FIG. 1 shows a schematic illustration of a cross-section through a heater assembly 100 in accordance with an example of the present disclosure, in which the heating element 110 is a porous layer. Referring to FIG. 1, the heater assembly 100 comprises: a heating element 110, a porous ceramic body 130, and electrical control circuitry (not shown for clarity).

The porous ceramic body 130 is configured to supply liquid aerosol-forming substrate to the heating element 110. Specifically, the porous ceramic body 130 is configured to transmit liquid aerosol-forming substrate from a liquid reservoir (not shown in FIG. 1 for clarity) to the heating element 110. The porous ceramic body 130 is configured to store some liquid aerosol-forming substrate before aerosolization by the heating element 110.

In this example embodiment, the porous ceramic body 130 is a cylindrical block The porous ceramic body 130 has a first end face and an opposing second end face. The first end face is a liquid absorption surface 134 and the second end face is a heating surface 133. In this example, the liquid absorption surface 134 and the heating surface 133 are both substantially flat surfaces. The porous ceramic body 130 also has a lateral face 131 extending between the liquid absorption surface 134 and the heating surface 133. The porous ceramic body 130 has a thickness defined between the liquid absorption surface 134 and the heating surface 133.

The porous ceramic body 130 comprises a plurality open-cell pores. The plurality of open-cell pores are interconnected to provide a fluid pathway for aerosol-generating liquid through the porous ceramic body 130.

The open pores are longitudinal pores which generally extend from the liquid absorption surface 134 to the heating surface 133 of the porous ceramic body. The pore size of the pores in the porous ceramic body 130 vary between the liquid absorption surface 134 and the heating surface 133.

The porous ceramic body 130 includes a heating end and a liquid absorption end, the heating surface 133 being disposed at the heating end, and the liquid absorption surface 134 being disposed at the liquid absorption end. The porous ceramic body includes a first average pore size at the liquid absorption end, and a second average pore size at the heating end. The first average pore size is greater than the second average pore size.

The first pore size at the liquid absorption end is about 150 micrometres. The second pore size at the heating end is about 20 micrometres. The pore size varies linearly between the first pore size and the second pore size to provide a pore size gradient between the liquid absorption end and the heating end of the porous ceramic body 130.

The pore structure and pore size gradient in the porous ceramic body 130 is achieved by etching the pores into a portion of silicon carbide.

The heater assembly 100 may be configured such that liquid can pass through the fluid pathway of the porous ceramic body 130 to the heating element 110, as depicted by arrows 170. The porous ceramic body 130 is configured for fluid 170 to pass from the liquid absorption surface 134 to the heating surface 133. The porous ceramic body 130 comprises a material which does not chemically interact with the liquid aerosol-forming substrate. The porous ceramic body 130 comprises ceramic. The porous ceramic body 130 comprises porous ceramic, such as but not limited to one or more of: Al2O3, ZrO2, Si3N4, SiC, Ti3AlC2, BN, AlN, SiO2, MgO, mica, diatomite, silicates, silicides, borides, glass. It will be appreciated that the porous ceramic body 130 may have a different shape or comprise a different material.

The heating element 110 is configured to heat a liquid aerosol-forming substrate to form an aerosol. The heating element 110 is configured to convert electrical energy into heat energy by material resistance of the heating element 110 to an electrical current.

The heating element 110 is arranged along the heating surface 133 of the porous ceramic body 130. The heating element 110 is in direct contact with the porous ceramic body 130.

The heating element 110 is a porous heating element. The heating element 110 extends to cover an area of the heating surface 133 of the porous ceramic body 130.

The heating element 110 has a first end face and an opposing second end face. The first end face is a liquid absorption surface 114 and the second end face is an outer surface 113. In this example, the liquid absorption surface 114 and the outer surface 113 are both substantially flat surfaces. The liquid absorption surface 114 of the heating element 110 is bonded to the heating surface 133 of the porous ceramic body 130 by an adhesive layer 115. In this example embodiment, the heating element 110 is a cylindrical block. The heating element 110 has a lateral face 111 extending between the liquid absorption surface 114 and the outer surface 113. The heating element 110 has a thickness defined between the liquid absorption surface 114 and the outer surface 113.

FIG. 2 is a schematic illustration of the interior of an aerosol-generating system 300 according to an example of the present disclosure. The aerosol-generating system 300 comprises two main components, a cartridge 301 and a main body part or aerosol-generating device 400. The cartridge 301 is removably connected to the aerosol-generating device 400. The aerosol-generating device 400 comprises a device housing 301 that contains a power supply in the form of a battery 402, which in this example is a rechargeable lithium ion battery, and control circuitry 403. The aerosol-generating system 300 is portable and has a size comparable to a conventional cigar or cigarette. A mouthpiece is arranged at a mouth end of the cartridge 301.

The cartridge 301 comprises a cartridge housing containing a heater assembly 100 and a liquid reservoir or liquid storage portion 303 for holding a liquid aerosol-forming substrate. Liquid aerosol-forming substrate is conveyed downwards from the liquid absorption surface 134 through the porous body to the heating element and vaporised aerosol-forming substrate is emitted from the heating surface 133 when electrical power is supplied to the heating element.

The cartridge 301 comprises one or more air inlets 304 formed in the cartridge housing 305 at a location along the length of the cartridge 301. An aerosol outlet 306 is located in the mouthpiece at the mouth end of the cartridge 301. The one or more air inlets 304 are in fluid communication with the aerosol outlet 306 to define an airflow pathway through the cartridge 301 of the aerosol-generating system 300. The airflow pathway flows from the one or more air inlets 304 to the heater assembly 100 in an airflow channel. The heater assembly 100 is arranged in fluid communication with the airflow pathway in the airflow channel. Air enters the one or more air inlets 304 and flows through the airflow channel past the heater assembly 100 in an average airflow direction.

In the example of FIG. 4, the liquid storage portion 303 is annular in cross-section and is arranged around a central sealed aerosol channel 307. Once the airflow pathway reaches the heater assembly 100, it is diverted upwards around the sides of the heater assembly 100 and flows through the aerosol channel 307 to the aerosol outlet 306.

The aerosol-generating system 300 is configured so that a user can puff or draw on the mouthpiece of the cartridge to draw aerosol into their mouth through the aerosol outlet 306. In operation, when a user puffs on the mouthpiece, air is drawn in through the one or more air inlets 304, along the airflow pathway through the airflow channel, past and around the heater assembly 100 and along the airflow pathway through the aerosol channel 307 to the aerosol outlet 306. The control circuitry 403 controls the supply of electrical power from the battery 402 to the cartridge 301 when the system is activated. This in turn controls the amount and properties of the vapour produced by the heater assembly 100. The control circuitry 403 includes an airflow sensor (not shown) and supplies electrical power to the heater assembly 100 when user puffs are detected by the airflow sensor. This type of control arrangement is well established in aerosol-generating systems such as inhalers and e-cigarettes. When a user puffs on the mouthpiece of the cartridge 301, the heater assembly 100 is activated and generates a vapour that is entrained in the airflow pathway. The vapour cools within the airflow pathway to form an aerosol, which is then drawn into the user's mouth through the aerosol outlet 306.

FIG. 5 is a schematic cross-sectional view of part of an aerosol-generating system 500 according to another example of the present disclosure showing an arrangement of a heater assembly 200 relative to an airflow pathway 520 within the aerosol-generating system 500. For simplicity, other components of the aerosol-generating system have been omitted from FIG. 5. The heater assembly 200 of FIG. 5 is identical to the heater assemblies 200 of FIGS. 1, 2 and 3. The aerosol-generating system 500 comprises a liquid storage portion 522 that holds a liquid aerosol-forming substrate in contact with the liquid absorption surface 202b of the porous body 202. Liquid aerosol-forming substrate is conveyed from the liquid storage portion 522 through the porous body 202 to the heating surface 202a, as indicated by arrows E. Vaporised aerosol-forming substrate is emitted through the porous heating element 204 from the heating surface 202a. As indicated by arrows F, the average vapour emission direction is substantially perpendicular to the heating surface 202a of the porous body 202.

In the example of FIG. 5, the heater assembly 200 is arranged below or to one side of the airflow channel or pathway 520, which airflow pathway 520 is defined by airflow channel walls 524. As viewed in FIG. 5, a left-hand end of the visible portion of the airflow pathway 520 receives airflow from an air inlet (not shown) and the right-hand end of the visible portion of the airflow pathway delivers airflow to an aerosol outlet (not shown). The heating surface 202a of the porous body 202 is arranged parallel to the airflow pathway 520 and faces into the airflow pathway 520. The heater assembly 200 is in fluid communication with the airflow pathway such that the airflow in the airflow pathway flows past the heater assembly 200 in an average airflow direction, as indicated by arrows G. The heater assembly 200 and airflow pathway 520 are arranged such that an angle θ between the average vapour emission direction F and the average airflow direction G is approximately 90 degrees, that is, at an angle θ substantially perpendicular to the average airflow direction G. The average vapour emission direction F has no speed or direction component that opposes the average airflow direction G and therefore any loss of momentum of the airflow is reduced. This reduces the tendency for recirculation and turbulence to occur in the airflow path 520 and the vapour is less likely to impinge on the internal surfaces of the airflow channel walls 524.

FIG. 6 is a schematic cross-sectional view of part of an aerosol-generating system 600 according to another example of the present disclosure showing another arrangement of a heater assembly 200 relative to an airflow pathway 620 within the aerosol-generating system 600. For simplicity, other components of the aerosol-generating system have been omitted from FIG. 6. The heater assembly 200 of FIG. 6 is identical to the heater assemblies 200 of FIGS. 2 and 3. The aerosol-generating system 600 comprises a liquid storage portion 622 that holds a liquid aerosol-forming substrate in contact with the liquid absorption surface 202b of the porous body 202. Liquid aerosol-forming substrate is conveyed from the liquid storage portion 622 through the porous body 202 to the heating surface 202a, as indicated by arrows E. Vaporised aerosol-forming substrate is emitted through the porous heating element 204 from the heating surface 202a. As indicated by arrows F, the average vapour emission direction is substantially perpendicular to the heating surface 202a of the porous body 202.

In the example of FIG. 6, the airflow channel or pathway 620 is split into first and second airflow pathway sections 620a and 620b which pass either side of the heater assembly 200. The first and second airflow pathway sections 620a and 620b combine downstream of the heater assembly 200 into a third airflow pathway section 620c. The first and second airflow pathway sections 620a and 620b receive airflow from one or more air inlets (not shown) and the third airflow pathway section 620c delivers airflow to an aerosol outlet (not shown). The airflow pathway 620 is defined by airflow channel walls 624. The heating surface 202a of the porous body 202 is arranged substantially perpendicular to the airflow pathway 620 and faces in a downstream direction of the airflow pathway 620. The heater assembly 200 is in fluid communication with the airflow pathway such that the airflow in the airflow pathway flows past the heater assembly 200 in an average airflow direction, as indicated by arrows G.

The heater assembly 200 and airflow pathway 220 are arranged such that an angle θ between the average vapour emission direction F and the average airflow direction G is less than 90 degrees. Upstream of the heating surface 202a of the porous body 202, the average airflow direction G past the heater assembly 200 is substantially the same as the vapour emission direction F. At the point along the airflow pathway 620 corresponding to the heating surface 202a the airflow pathway 620 starts to narrow or taper inwards, at which point the average airflow direction G past the heater assembly 200 changes to an angle θ relative to the vapour emission direction F of approximately 45 degrees. Downstream of the heating surface 202a of the porous body 202 in the third airflow pathway section 620c, the average airflow direction G of the combined airflow is again substantially the same as the vapour emission direction F. It will be appreciated that the narrowing or tapering of the airflow pathway 620 could be omitted. In which case, the average airflow direction G past the heater assembly 100 would be substantially the same as the vapour emission direction F.

FIGS. 7 and 8 show a schematic illustration of an example of a heater assembly 700 for an aerosol-generating system. The heater assembly includes a heating element 710 and a porous body 720.

The heating element 710 is configured to vaporise an aerosol-forming substrate, such as a liquid aerosol-forming substrate, to form an aerosol. The heating element 710 is configured to convert electrical energy into heat energy by material resistance of the heating element 710 to an electrical current. In this example embodiment, the heating element 710 is in direct contact with the porous ceramic body 720.

The porous body 720 is configured to convey the liquid aerosol-forming substrate to the heating element 710. In other words, the porous body 720 supplies the liquid aerosol-forming substrate to the heating element 710.

The porous body 720 has a first end face and an opposing second end face. The first end face is a liquid absorption surface 730 and the second end face is a heating surface 740. In this example, the liquid absorption surface 730 and the heating surface 740 are both substantially flat surfaces. The porous body 720 also has a plurality of lateral faces extending between the liquid absorption surface 730 and the heating surface 740.

In this example, as will be discussed in more detail below, the porous body 720 has a first lateral face 750 opposing a second lateral face 760, and a third lateral face 770 opposing a fourth lateral face 780.

The porous body 720 comprises a plurality of pores. The plurality of pores are interconnected to provide a fluid pathway for liquid aerosol-forming substrate through the porous body 720, from the liquid absorption surface 730 to the heating surface 740. The porous body 720 is formed from a material that does not chemically interact with the liquid aerosol-forming substrate. In this example, the porous body 720 is a porous ceramic body and may be formed from, for example, Ca₂SiO₃ or SiO₂ (or Ca₂SiO₃ and SiO₂).

The heating element 710 is located on and bonded to the heating surface 740 of the porous body 720. In the example of FIGS. 7 and 8, the heating element 710 is a porous film that extends across substantially all of the heating surface 740.

The liquid absorption surface 730 of the porous body 720 has an area that is different to an area of the heating surface 740 of the porous body 720. Specifically, in the example of FIGS. 7 and 8, the area of the heating surface 740 is less than the area of the liquid absorption surface 730.

In the example of FIGS. 7 and 8, the heating surface 740 has smaller area than the liquid absorption surface 730 because the length of the heating surface 740 is less than the length of the liquid absorption surface 730. In addition, or alternatively, in another example, the heating surface 740 may have a smaller area than the liquid absorption surface 730 because the width of the heating surface 740 is less than the width of the liquid absorption surface 730.

In the example of FIGS. 7 and 8, the porous body 720 is shaped as a trapezoid prism. With the porous body 720 having a trapezoid prism shape, the first lateral face 750 and the second lateral face 760 both have a trapezium shape, specifically an isosceles trapezoid, the third lateral face 770 and the fourth lateral face 780 both have a rectangle shape, and the liquid absorption surface 730 and the heating surface 740 both have a rectangle shape. In another example, the liquid absorption surface 730 and the heating surface 740 may have a square shape.

The porous body 720 tapers from the liquid absorption surface 730 towards the heating surface 740. In other words, the cross-sectional area of the porous body 720 gradually becomes smaller from the liquid absorption surface 730 towards the heating surface 740. In the example of FIGS. 7 and 8, the length of the porous body 720 decreases from the liquid absorption surface 730 towards the heating surface 740 which causes the tapering.

FIG. 9 shows a heater assembly 900 for use in an aerosol-generating system. The heater assembly 900 comprises a heating element 910 for vaporising a liquid aerosol-forming substrate. The heater assembly 900 also comprises a porous body 920 for conveying the liquid aerosol-forming substrate to the heating element 910. The porous body 920 has a liquid absorption surface 921 and an opposed heating surface 922. The heating element 910 is located on the heating surface 922 of the porous body 920. The porous body 920 can be made from any suitable ceramic material such as the materials discussed in any of the examples above.

The heating surface 922 of the porous body 920 is curved. In particular, the heating surface 922 of the porous body 920 is convexly curved in a single transverse direction (the first transverse direction).

The porous body 920 is prismatic in shape. When viewing a longitudinal cross-section perpendicular to the direction of curvature of the porous body 920, the heating surface 922 of the porous body 920 is shown as an arc. The porous body 920 has two longitudinal planes of symmetry.

The heating surface 922 of the porous body 920 has a width 923 in the first transverse direction substantially the same as the width of the porous body 920 in the first transverse direction, and substantially the same as the width of the heater assembly 900 in the first transverse direction. The heating surface 920 of the porous body 920 has a width of about 5 millimetres in the first transverse direction.

The heating surface 922 of the porous body 920 has a length or thickness 924 of about 1 millimetre. The porous body 920 has a length or thickness 925 of about 3 millimetres.

The heating surface 922 of the porous body has a of curvature of about 3.6 millimetres. The heating surface 922 of the porous body has a surface area of about 28 square millimetres.

The porous body 920 comprises four longitudinal surfaces or side walls extending from the liquid absorption surface 921 to the heating surface 922. The four side walls are substantially perpendicular to the liquid absorption surface 921, which is substantially flat. The liquid absorption surface 921 is square in shape.

The heating element 910 is a resistive heating element 910 and is curved. In particular, the curvature of the heating element 910 is substantially the same as the curvature of the heating surface 922 of the porous body 920. As such, the heating element 910 is also convexly curved in a single transverse direction.

The heating element 910 is located directly on the heating surface 922 of the porous body 920. The heating element 910 extends across a majority of the heating surface 922 of the porous body 920. Substantially the entirety of the heating element 910 is in contact with the heating surface 922 of the porous body 920.

FIG. 10 shows a schematic cross-sectional view of a heater assembly 1000 for an aerosol-generating system according to an example of the present disclosure. The heater assembly 1000 comprises: a heating element 1010, a thermally insulating layer 1020 and a porous body 1030. The porous body 1030 is configured to supply liquid aerosol-forming substrate to the heating element 1010. Specifically, the porous body 1030 is configured to transmit liquid aerosol-forming substrate from a liquid reservoir (not shown) to the heating element 1010. The porous body 1030 is configured to store some liquid aerosol-forming substrate before aerosolization by the heating element 1010.

The porous body 1030 is a rectangular block and has a first end face and an opposing second end face. The first end face is a liquid absorption surface 1034 and the second end face is a heating surface 1033. In this example, the liquid absorption surface 1034 and the heating surface 1033 are both substantially flat surfaces. The porous body 1030 also has a plurality of lateral faces extending between the liquid absorption surface 1034 and the heating surface 1033. The porous body 1030 has a first lateral face 1031 opposing a second lateral face 1032, and a third lateral face (not shown) opposing a fourth lateral face (not shown). The porous body 1030 has a thickness defined between the liquid absorption surface 1034 and the heating surface 1033.

The porous body 1030 comprises a plurality of open-cell pores. The plurality of open-cell pores are interconnected to provide a fluid pathway for aerosol-generating liquid through the porous ceramic body 1030. The heater assembly 1000 may be configured such that liquid can pass through the fluid pathway of the porous body 1030 to the heating element 1010, as depicted by arrows 1070. The porous body 1030 is configured for fluid 1070 to pass from the liquid absorption side 1034 to the heating surface 1033. The porous body 1030 comprises a material which does not chemically interact with the liquid aerosol-forming substrate. The porous body 1030 comprises ceramic. The porous body 1030 comprises porous ceramic, such as but not limited to one or more of: Al₂O₃, ZrO₂, Si₃N₄, SiC, Ti₃AlC₂, BN, AlN, SiO₂, MgO, mica, diatomite, silicates, silicides, borides. Alternatively, the porous body 830 may comprise porous glass. It will be appreciated that the porous body 1030 may have a different shape or comprise a different material.

The heating element 1010 is configured to heat a liquid aerosol-forming substrate to form an aerosol. The heating element 1010 is configured to convert electrical energy into heat energy by material resistance of the heating element 1010 to an electrical current.

The heating element 1010 comprises a track defining a path across a heating surface 1023 of the thermally insulating layer 1020. The heating element 1010 defines a serpentine or an electrically parallel track shape across the heating surface 1023 of the thermally insulating layer 1020. Three cross-sections through portions of the track of the heating element 1010 are shown in FIG. 12. The plurality of track portions are arranged with distances between at least two of the plurality of track portions 1218, 1219 in the range 200 to 300 micrometres. The track portions are evenly spaced. It will be appreciated that distances between at least two of the plurality of track portions 1218, 1219 may not be equal.

The heating element 1010 is elongate and comprises metal, such as but not limited to stainless steel, Ni—Cr alloy, NiCrAlY alloy, FeCrAl alloys (e.g., Kanthal), FeCrAlY alloys, Fe₃Al alloy, Ni₃Al alloy, NiAl alloy, and CuNi alloys. It will be appreciated that the heating element 810 may have a different shape or comprise a different material.

The heating element 1010 is arranged along an outer surface of the thermally insulating layer 1020. The heating element 1010 is in direct contact with the thermally insulating layer 1020.

The thermally insulating layer 1020 is arranged to enhance thermal insulation between the heating element 1010 and the porous body 1030. The thermally insulating layer 1020 is arranged to extend across at least a portion of the heating element 1010 to thermally insulate the heating element 1010 from the porous body 1030. The thermally insulating layer 1020 is configured to reduce heat dissipation through the porous body 1030, so as to enhance energy efficiency by reducing energy losses.

The thermally insulating layer 1020 is planar and has a size and shape configured to extend across the electrical heating element 1010. The thermally insulating layer 1020 is configured to entirely extend across a surface of the heating element 1010. The thermally insulating layer 1020 is configured to substantially cover the porous body 1030 below the thermally insulating layer 1020.

The thermally insulating layer 1020 has a first end face 1024 and an opposing second end face 1023. In this example, the first 1024 and second 1023 end faces are both substantially flat surfaces. The first end face 1024 of the thermally insulating layer 820 is in direct contact with the porous ceramic body 1030. The second end face 1023 of the thermally insulating layer 1020 is in direct contact with the heating element 1010.

The thermally insulating layer 1020 has a thickness defined between the first end face 1024 and the second end face 1023. The thickness of the thermally insulating layer 1020 is less than the thickness of the porous body 1030. The thermally insulating layer 1020 may have a thickness between 0.1 mm and 2 mm, preferably between 0.5 mm and 1.5 mm.

The thermally insulating layer 1020 comprises a material having a low thermal conductivity. The thermally insulating layer 1020 comprises or consists of a material with a lower thermal conductivity than the porous ceramic body 1030. The thermally insulating layer 1020 may have a higher porosity than the porous ceramic body 1030. The thermally insulating layer 1020 may comprise a material such as one or more of: alumina, zirconia, zirconia with magnesium oxide, glass ceramic, quartz, a porous polymer. It will be appreciated that the thermally insulating layer 1020 may have a different shape or comprise a different material.

FIG. 11 shows a schematic cross-sectional view of another example heater assembly 1001 for an aerosol-generating system. The heater assembly 1001 of FIG. 11 is the same as the heater assembly 1000 of FIG. 11, with the exception that the heating element 1015 comprises a porous heating element. The porous ceramic body 1030, and the thermally insulating layer 1020 are as described in relation to the heater assembly 1000 of FIG. 10 and like reference numerals have been used to label like components.

The heating element 1015 extends to cover an area of the second end face 1023 of the thermally insulating layer 1020. The heating element 1015 has a liquid absorption surface 1014 and a heating surface 1013. In this example, the liquid absorption surface 1014 of the heating element 1015 and the heating surface 1013 of the heating element 1015 are both substantially flat surfaces. The liquid absorption surface 1014 of the heating element 1015 is in direct contact with the thermally insulating layer 1020.

Referring to FIGS. 12 (a) to 12 (c), there are shown schematic illustrations of different heating elements 110 for an aerosol-generating system. Each heating element 110 comprises a plurality of tracks or track portions 117 arranged electrically in parallel. By being arranged electrically in parallel, current flow is split into separate parallel flow paths. The flow paths are subsequently re-combined.

In the heating elements 110 of FIGS. 12 (a) to 12 (c), each heating element 110 comprises a first connecting pad 113 and a second connecting pad 114. The first and second connecting pads 113, 114 are configured to allow connection to an external circuit. An aperture or plurality of apertures 115 in the heating element 110 separate each track 117. Each heating element 110 comprises a diverging portion, in which current is split from the first connecting pad 113 into tracks 117 which define electrically parallel paths. Each heating element 110 comprises a converging portion, in which current is combined from tracks 117 which define electrically parallel paths, into the second connecting pad 114.

Various different arrangements are possible of tracks or track portions arranged electrically in parallel. In FIG. 12 (a), four tracks 117 are separated by three apertures 115 to define four electrically parallel paths. In FIG. 12 (b), six track portions 117 are separated by one aperture 115 to define two electrically parallel paths. In FIG. 12 (b), each electrically parallel path defines a serpentine path between the first connecting pad 113 and the second connecting pad 114. In FIG. 12 (c), eight track portions 117 are separated by four apertures 115 to define four pairs of electrically parallel paths. Each pair of electrically parallel path in FIG. 12 (c) is separated by an intermediate connection 116, of which three are shown in FIG. 12 (c).

By having tracks or track portions arranged electrically in parallel, if one track portion is defective, current can be redistributed and can still flow through the heating element 110, i.e. the electrical connection between the first connecting pad 113 and the second connecting pad 114 is not broken. This has the advantage of increasing the number of puffs before full failure of the heater, and potentially increasing the heater lifetime up to the lifetime of the device. In contrast, in a simple serpentine heater defining a single electrical path between a first connecting pad 113 and a second connecting pad 114, if a part of the serpentine heating element is broken, then the heating element will stop working due to an increase in local resistance at the breakage or defect point. A defect in a simple serpentine heater causes an increase in local resistance. An increase in local resistance causes increased power dissipation. Increased power dissipation in turn increases the resistance until breakage.

The inventors have also identified that the parallel tracks or track portions arranged electrically in parallel, explained with reference to FIGS. 12 (a) to (c), has a surprising additional advantage. In such an arrangement, in case of breakage of one track portion, the heating element will still operate and can, for an initial transitory period, operate in an advantageous way, because the breakage of one track or track portion would result in a higher energy density on the remaining tracks or track portions. In such a case, the same power would still be provided but on a smaller area, so the throughput would be increased. While such a breakage causing an increase in current on unbroken tracks or track portions can eventually degrade the user experience, the device or cartridge can include a mechanism to alert the user about possible future below optimal performance of the heater assembly.

Such a mechanism relies on the following principles. The total electrical resistance of the heating element depends on the following factors:
1) the number of heating tracks in parallel (more parallel tracks decrease the total resistance);
2) the cross-sectional area (width or thickness (or width and thickness)) of the parallel heating tracks (a higher cross-sectional area leads to a lower resistance);
3) the length of the parallel heating tracks (longer tracks have a higher resistance);
4) if the heating element is porous, tuning the porosity of heating element (higher porosity increases resistance);
5) particular chemical or material composition (e.g. alloys by doping).

The overall total heating element resistance Rₜₒₜ of an arrangement of a number of heating tracks or track portions (i) arranged in parallel such that electric current in at least two neighbouring tracks or track portions flows in the same direction, Rᵢ is set out in equation 4:

$$\frac{1}{{R}_{tot}} = {\sum }_{i}^{n} \frac{1}{{R}_{i}} \qquad ( 4 )$$

where n is the total number of heating tracks arranged electrically in parallel.

The behavior of a parallel track heating element when one heating track fails can be considered with reference to a heating element with 4 parallel heating tracks, for example as shown in FIG. 12 (a). The heating tracks each have a resistance of 3 Ohms. The total resistance of the heating element is 0.75 Ohms, calculated using equation 4.

When one heating track starts to fail, the resistance of the failing heating track increases. The total resistance of the heating element also starts to increase, following a linear relationship with the failing heating track resistance. However, as the heating track resistance continues to increase, the heating element resistance asymptotes to a constant resistance value. At this constant resistance value, the influence of the failing heating track on the heating element resistance is capped. In this example where unbroken heating tracks each have a resistance of 3 Ohms, the total resistance of the heating element that asymptotes to 1 Ohm when the failed track can be considered as an open circuit (i.e. no more current can flow through it). When one track breaks in this example, only three tracks remain for the purpose of calculating the total heating element resistance.

To consider the behavior of such a heating element, a supply voltage of 3.5 Volts and target power of 5.5 Watts are considered. In this example, unbroken parallel heating tracks remain with their initial resistance of 3 Ohms. In the failing track, the total maximum current decreases with increasing resistance. In the failing track, current decreases to zero once broken. The current through the unbroken parallel tracks remains substantially constant as the resistance of the failing track increases (if resistance change due to temperature increase is ignored).

A similar behavior is observed for the maximum power generation. Less total power is generated once a heating track has failed. However, in this example, the maximum power remains higher than the target of 5.5 Watts despite failure of one of the heating tracks.

The device or cartridge may also be configured to extend the life of the parallel track heating element. The aerosol-generating device or system may comprise control circuitry. The control circuitry may be configured to, after detecting the failure of a heating track for example by a feedback loop, adjust the power fed to the heater. The control circuitry may be configured to provide a pulse width modulation (“PWM”) signal to control the power fed to the heater. The control circuitry may adjust the power fed to the heater by adjusting the duty cycle of the pulse width modulation signal. In an example, control circuitry may be configured to have a duty cycle at 33.7 percent when the heating tracks are in a normal condition. The duty cycle may increase to 44.9 percent when one of the heating tracks has failed. When one of the heating tracks fails, the power density (heating power generated by surface area) increases, enhancing the thermal efficiency of the heater body. Therefore, the proper operation of the heater is not jeopardized with one failed heating track. A similar result occurs if a second heating track breaks. The control circuitry may be configured such that the duty cycle further increases (to 67.4 percent in the current example). Thus, a heating element with four parallel heating tracks can still operate with the nominal condition of 5.5 Watts even if two of these heating tracks are broken, since the duty cycle remains below 100 percent.

The control circuitry may be configured such that, based on the change of nominal total resistance of the heating element once a parallel heating track has failed, it is possible for the control circuitry to assess the state of the heating element (i.e., number of heating tracks which have failed). The control circuitry may be configured such that, after a predefined number of heating track(s) have failed, the device can tell the user that the heater assembly should be changed.

Referring to FIGS. 13 (a) and 13 (b), there are shown schematic illustrations of current flow 1309 around a corner of a heating element track.

FIG. 13 (a) is a schematic illustration of current flow 1309 around a known heating element in which a track portion defines a path having a bend, the inner edge of the bend having a sharp corner. In such a track, current flow depicted by arrows 1309, which follows a path of least resistance, is concentrated (i.e., there is an increase in current density). This concentration occurs at an inner edge of the corner. Current concentration can increases the local temperature, and can lead to the presence of hot spot at the corner. A hot spot is disadvantageous, as it can affect the efficiency and reliability of the heating element. A hot spot occurs despite the potential for local resistivity of the heater track material to increase due to a local increase in temperature (which would direct current flow away to a path of lower resistance).

FIG. 13 (b) is a schematic illustration of current flow 1309 around a heating element in which a track portion 1317 defines a path having a bend, the inner edge of the bend being curved. In such a track 1317, current flow 1309 does not form a local hot spot.

In contrast to the track shape shown in FIG. 13 (a), current flow 1309 in the smoother curved track portion 1317 as shown in FIG. 13 (b) remains more evenly distributed across the heating track 1317, as depicted by dashed arrows 1309. Current flow 1309 is guided to flow more evenly, to avoid a concentration of current at any point. This in turn limits hot spot creation. The heater track 1317 may have a gradient of electrical resistivity perpendicular to current flow in a corner or corners, such that the electrical resistivity is higher at an inner part of the corner and lower at an outer part of the corner. Such a gradient is beneficial to counterbalance localized high current density and reduce hot spot creation.

1.-15. (canceled)

16. A heater assembly for an aerosol-generating system, the heater assembly comprising:
a heating element configured to vaporise a liquid aerosol-forming substrate; and
a porous ceramic body configured to convey the liquid aerosol-forming substrate to the heating element, the porous ceramic body having a liquid absorption surface and a heating surface,
the heating element being located on and bonded to the heating surface of the porous ceramic body.

17. The heater assembly according to claim 16, wherein the heating element is in contact with the heating surface of the porous ceramic body, and the heating element and the heating surface of the porous ceramic body are bonded by virtue of this contact.

18. The heater assembly according to claim 16, wherein the heating element and the heating surface of the porous ceramic body are bonded by an intervening layer located between the heating element and the heating surface of the porous ceramic body

19. The heater assembly according to claim 16, wherein the heating element is a porous ceramic heating element.

20. The heater assembly according to claim 19, wherein the heating element is a doped portion of the porous ceramic heating element.

21. The heater assembly according to claim 16, wherein the heating element is a metallic heating element.

22. The heater assembly according to claim 16, wherein the heating element is a metallic heating track.

23. The heater assembly according to claim 16, wherein the heating element is deposited on the porous ceramic body by thick film techniques.

24. The heater assembly according to claim 16, wherein the heating element is deposited on the porous ceramic body by screen printing, inkjet printing, aerosol-jet printing, or laser direct structuring.

25. The heater assembly according to claim 16, wherein the heating element is deposited by thin film techniques.

26. The heater assembly according to claim 16, wherein the heating element is deposited by physical vapor deposition or chemical vapor deposition.

27. The heater assembly according to claim 16, wherein the heating element and the porous ceramic body are bonded by molding.

28. The heater assembly according to claim 16, wherein the heating element and the porous ceramic body are formed separately and assembled to form the heater assembly.

29. The heater assembly according to claim 16, wherein the liquid absorption surface of the porous ceramic body has an area that is different from an area of the heating surface of the porous body.

30. The heater assembly according to claim 16, wherein the heating surface of the porous body is convex in one or both of a first transverse direction and a second transverse direction, the first transverse direction being orthogonal to the second transverse direction.

31. The heater assembly according to claim 30, wherein the heating surface of the porous body is convex in a single transverse direction.

32. The heater assembly according to claim 16,
further comprising a thermally insulating layer having a lower thermal conductivity than the porous body,
wherein the thermally insulating layer is disposed between and is in contact with each of the porous body and the heating element, and the thermally insulating layer is configured to reduce heat transfer from the heating element to the porous body.

33. A method of manufacturing a heater assembly for an aerosol-generating system, the method comprising:
forming a heating element configured for vaporising a liquid aerosol-forming substrate;
forming a porous ceramic body configured for conveying the liquid aerosol-forming substrate to the heating element, the porous ceramic body having a liquid absorption surface and a heating surface; and
bonding the heating element on the heating surface of the porous ceramic body.

34. An aerosol-generating system, comprising:
the heater assembly according to claim 16, wherein the heating element is fluid-permeable such that vapour is emitted from the heater assembly in an average vapour emission direction; and
an air inlet and an aerosol outlet, the air inlet being in fluid communication with the aerosol outlet to define an airflow pathway through the aerosol-generating system,
wherein the heater assembly is arranged in fluid communication with the airflow pathway such that air flows past the heater assembly in an average airflow direction, and
wherein the heater assembly and airflow pathway are arranged such that an angle between an average vapour emission direction and the average airflow direction is less than 135 degrees.
